(19) United States
(12) Patent Application Publication
(10) Pub. No.: US 2002/0004197 A1
(43) Pub. Date: Jan. 10, 2002

(54) Method for generating a pathway reporter system

(75) Inventors: Teri Melese, Mountain View, CA (US); Edward L. Perkins, Mountain View, CA (US); Stuart Tugendreich, Mountain View, CA (US)

Correspondence Address: ROBINS & PASTERNAK LLP, Suite 200, 90 Middlefield Road, Menlo Park, CA, 94025, US

(73) Assignee: Iconix Pharmaceuticals, Inc.

(21) Appl. No.: 09/941,405

(22) Filed: Aug. 28, 2001

Related U.S. Application Data
(63) Continuation of application No. 09/475,412, filed on Dec. 30, 1999, now abandoned.
(60) Provisional application No. 60/114,399, filed on Dec. 31, 1998.

(51) Int. Cl. C12Q 1/00; C12Q 1/68; C12N 15/63

(52) U.S. Cl. 435/4; 435/6; 435/483

(57) ABSTRACT

Methods and nucleic acid constructs for testing and validating activity reporter cells, using a plurality of host cell genes, are described.

### RELATED APPLICATIONS

This application claims priority from U.S. Ser. No. 60/114,399, filed Dec. 31, 1998, incorporated herein by reference in full.

### FIELD OF THE INVENTION

This invention relates to the fields of microbiology and drug discovery. More particularly, the invention relates to methods for preparing assay vehicles for investigating gene function.

### BACKGROUND OF THE INVENTION

It is often difficult to determine the function of a gene in an organism, as many genes interact in complex webs with overlapping pathways. One can study genes by isolating nucleic acids and transferring them to a foreign host cell, which is less likely to respond to the transferred gene, but may still exhibit some response. However, some genes fail to exhibit any detectable change in the host cell, for example due to alternate metabolic or signaling pathways available to the host cell.

Screening for therapeutically useful compounds has commonly used biochemical screening and/or whole cell screening, in which cells are contacted with a compound under conditions which are believed to be relevant to the intended use of the compound and the cells are monitored for a particular readout which is indicative of an active compound. However, it is often difficult to design an assay that provides a useful readout. For example, one can arrange an assay for an isolated surface receptor that determines when a test compound binds to the target receptor, but simple binding does not indicate that the receptor is also activated or inhibited by the test compound.

### SUMMARY OF THE INVENTION

We have now invented a method for modifying host cells having a transfected gene so that a detectable phenotype is produced.

One aspect of the invention is a method for preparing a plurality of assays, by transforming a plurality of host cells with nucleic acid constructs comprising a host cell gene linked to a detectable reporter (and optionally to a selectable marker and/or an affinity label) to provide a plurality of reporter cells, and transforming the reporter cells with a heterologous gene to provide a plurality of different transformed reporter cells. The transformed reporter cells are then selected for modulation of the detectable label expression (or affinity label expression, or selection due to the selectable marker) as a result of the heterologous gene activity. Preferably, the transformed reporter cells are selected based on modulation that differs under different selected culture conditions.

Another aspect of the invention is a method for examining the activity of a heterologous gene in a host cell, by transforming a plurality of host cells containing said heterologous gene with a plurality of nucleic acid constructs, each said construct comprising a different host gene operatively linked to a detectable label, and optionally to a selectable marker and an affinity label. The resulting transformants are subjected to variations in culture conditions (for example, changes in temperature, nutrients, crowding, chemicals, proteins, and the like), and transformants that exhibit a change in label expression as a function of culture conditions are selected. The method enables one to determine all host genes that interact with the heterologous gene (or its product).

Another aspect of the invention is a nucleic acid construct useful in the method of the invention, comprising a host cell gene, a detectable label, and optionally a selectable marker and affinity label. The construct is preferably flanked by recombinase recognition sites, and preferably further comprises appropriate maintenance and replication sequences sufficient for propagation in cloning and expression hosts.

Another aspect of the invention is a method for determining the biological effect of a compound, by contacting a panel of host cells with the compound, and determining the change (if any) in expression of a detectable label, wherein each host cell comprises a heterologous gene and a detectable label, wherein the label is expressed in response to activation of a host cell gene by the heterologous gene (or its product).

Another aspect of the invention is a method for predicting the activity of a heterologous gene, by providing a panel of reporter cells as described above, transforming the reporter cells with a plurality of different heterologous genes of known function, and determining which heterologous genes are associated with activity in the reporter cells. The unknown gene is also transformed into a plurality of reporter cells, and its function determined by similarity to a gene of known function, where said similarity is based on the reporter cells activated by said genes.

### DETAILED DESCRIPTION

Definitions:

The term “essential gene” as used herein refers to a gene whose function is required for viability of its host, i.e., the host cell dies if the essential gene function is lost.

The term “detectable label” as used herein generally refers to a gene that encodes a product which can be detected by optical or fluorescent techniques, or by performing simple enzymatic assays (for example, lacZ). Detectable labels preferably exhibit characteristic spectra that permits their use in FACS and/or other optical-based sorting systems.

The term “affinity marker” refers to a gene encoding a protein, polypeptide, or epitope having binding characteristics that permit one to sort the protein by means of an affinity column. Exemplary affinity markers include, without limitation, HA, avidin, biotin, streptavidin, and the like.

The term “selectable marker” as used herein refers to a gene encoding a protein essential to survival of the host cell (or alternatively, capable of killing the host cell under specific conditions). Suitable selectable markers include HIS3, thymidine kinase, and the like.

The terms “DNA array” and “microarray” are used interchangeably to refer to devices capable of detecting the presence of one or more nucleic acid sequences in a sample, such as, for example, the DNA chip technology commercialized by Affymetrix. “Array” as used herein refers to a plurality of objects arranged in a pattern, in which different objects are distinguished by their position in the pattern. Arrays are often set out in two-dimensional grids, but may be arranged in any way desired.

The term “ARC” or “activity reporter cell” refers to a host cell containing a heterologous gene, in which the heterologous gene produces a detectable phenotype in the host cell. The phenotype varies in response to an additional factor, which can be environmental (for example, temperature, cell contact, and the like), chemical, or the presence of additional heterologous genes in the host.

The term “recombinase” refers to an enzyme which cleaves nucleic acids at a specific recognition site or sequence, facilitating integration of a nucleic acid into a host cell genome. Exemplary recombinases include, without limitation, cre.

General Method:

The technology for using yeast as a surrogate host to express foreign proteins is now well established. However, there still exists a need for methods to assess the genome-wide impact of a protein on the host cell's physiology, particularly for proteins of unknown function. The instant invention (PRIYSM) is designed to report the effect of heterologous gene expression on cellular pathways in the surrogate host, and represents an improvement over technologies based on DNA microarrays (“chips”). DNA chips tend to be static, and to provide a readout at only a single point in time (or at selected points), whereas the method of the invention is capable of providing a continuous readout. Information derived using the method of the invention can be used to design genetic tests to establish relationships between multiple heterologous genes and compounds.

The application of PRIYSM for reporting the genomic effects of heterologous gene expression in a surrogate host involves constructing a yeast genomic library in a transposon tagging system (for example, an E. coli based transposon tagging system), transposon tagging the yeast genomic library, introducing the transposon-tagged gene fusion library constructs into yeast, screening for appropriate reporter-linked cellular readouts, and applying the PRIYSM technology to globally monitor the effects of heterologous gene expression.

In the initial stage, a library is constructed consisting of target nucleic acids (for example, host genomic DNA fragments) of approximately 5 Kb in size cloned into a modified shuttle vector (e.g., an E. coli/yeast shuttle vector). The shuttle vector contains all the required factors necessary for plasmid maintenance in E. coli and some required for the host, for example an E. coli replication origin and antibiotic resistance marker, as well as a yeast centromere and a yeast autonomous replication sequence. The eukaryotic host genomic fragments are cloned into the plasmid, and the library propagated in an E. coli host. The eukaryotic host genomic fragments are inserted flanked by loxP sites if cre recombinase is to be used, or other sites recognized by the recombinase enzyme to be used if other than cre. The library is constructed such that there is a sufficient number of cloned transformants to guarantee a probability greater than 99% that complete coverage of the eukaryotic host genome will be included. Where the eukaryotic host is yeast, this is approximately 20,000 recombinants. The E. coli host is selected to provide all the genetic factors necessary for transposon tagging of the eukaryotic host genomic fragments, as well as the necessary enzymes for catalyzing transposition and resolution (provided in trans). Examples of these types of yeast transposon tagging systems include the Tn10 based “lambda hopping system” and the Tn3 transposon tagging system (O. Huisman et al., Genetics (1987) 116(2):191-99; P. Ross-Macdonald et al., Proc Natl Acad Sci USA (1997) 94:190-95).

Ross-Macdonald et al. (supra) described a transposon tagging system employing Tn3, a green fluorescent protein (GFP) and a hemagglutin antigen epitope tag (HA) adjacent to a yeast selectable marker. When this element transposes in-frame to a yeast gene, a recombinant fusion protein is generated consisting of the yeast gene product fused to the GFP-HA element. The instant method in general employs a detectable label (such as, for example, GFP or a variant thereof), an affinity marker or antigen (such as, for example, HA), and further includes a selection marker, such as a yeast URA3 gene fused in-frame, such that a functional URA3 protein is produced only if inserted in-frame into a yeast gene.

The resulting transposon construct is then transposed into the host genome fragment library, following standard protocols. Successful transpositions introduce a yeast selectable marker into the plasmids. Following the subsequent purification of the host genomic library (containing the random insertion of transposable elements), the library is transformed into a matching eukaryotic host (e.g., yeast), utilizing the selectable marker inserted into the transposon element to generate potential eukaryotic gene fusion reporter-linked strains, where the gene fusions are propagated as autonomous replicating DNA molecules. Approximately 100,000 transformants are typically sufficient. The transformants are isolated and inoculated into microtiter dishes to serve as a first layer for arraying the possible reporter-linked strains. Utilizing microarray technology, the transformants can be “printed” onto soft agar growth media to form intermediate “chip” arrays. These intermediate arrays are then exposed to various stress conditions, whether by varying the environment, or by providing a varying environment as part of the “chip” (e.g., by establishing one or more chemical concentration gradients across the chip). Host cells that contain gene fusions that respond to the various conditions are identified as those that demonstrate an increase or decrease in fusion gene expression (determined, for example, by fluorescence microscopy utilizing the GFP construct). The identified host cells are then re-arrayed in order to generate a panel of gene fusion constructs that can globally monitor the effect of heterologous gene expression on cellular pathways in the surrogate host. Finally, the reporter gene fusions can be integrated into the host genome by transforming the cells with a second plasmid expressing the appropriate recombinase (e.g., cre recombinase). The recombinase facilitates integration of the gene fusion into the host genome.

The resulting panel is useful for examining activity reporter cells (ARCs), which contain one or more heterologous genes which produce a phenotype in the host cell (where the phenotype depends on the biological activity of the heterologous gene). See U.S. Ser. No. 09/187,918, filed Nov. 7, 1998, incorporated herein by reference in full. The reporter panel can also be generated “manually” by isolating the promoters from some or all of the host's genes by PCR, and individually linking them to the reporter gene. Since the heterologous gene may affect a variety of host genes, the panel of the invention provides a means for assaying that activity. Once the PRIYSM panel is established, a surrogate host containing the heterologous gene can be easily mass mated to the panel of reporter linked constructs, or otherwise transformed with the reporter constructs. The resulting mated host cells can be arrayed again, for example into soft agar, and the heterologous gene expressed. Again, fluorescence microscopy can be used to identify reporter constructs whose expression is altered by the heterologous gene. This results in a genetic network of cell-based reporters for each heterologous gene tested. Alternatively, the panel itself can be transfected with a heterologous gene (or construct) directly, thus forming ARCs in situ. Such transformation can be performed on the panel as a pool of cells or arranged in an array.

Additionally, reporters can be selected directly in ARCs, including ARCs that fail to demonstrate an obvious phenotype. For this, the constructed gene fusion library is transformed directly into the host strain containing the heterologous gene. Upon expression of the heterologous gene, the affected reporters can easily be identified either by direct selection for or against URA3 function (including, for example, identification using a DNA array), or can be sorted using FACS or similar technologies, employing the GFP. The identified reporters can then be arrayed to generate a PRIYSM panel specific for each heterologous gene. This approach circumvents the requirement of a growth interference/complementation phenotype, and directly establishes multiple reporter linked assays for each heterologous gene. Finally, the identified reporters can be integrated into the host genome by the cre-lox method set forth above.

The method of the invention can also be applied to essential genes, by omitting any integration step. Integration into an essential gene can cause loss of function, with resulting death of the host cell. However, in the present method, the PRIYSM constructs can be used in plasmid form, without requiring integration into the host cell genome.

In contrast to current array technology, which provides only a readout at a given point in time, the method of the invention can provide continuous data, a physiological readout of a set of chosen cellular pathways, without relying on a growth readout. Further, PRIYSM is genetically tractable, and extends the use of global reporting. The three-part fusion constructs employed in the invention (e.g., GFP-URA3-HA) enables one to use any fusion construct whose expression is modulated or altered by a heterologous gene as a functional tool, using selection based upon prototrophy (or by cell sorting using the marker) provides multiple entry points for ARC expansion (for example, cloning more members of a protein family which has been found to induce a particular reporter) using chemicals and/or other expressed genes. More importantly, this expansion can be directed to any or all of the entry points, allowing a greater degree of precision for ARC expansion. For example, if the initial PRIYSM analysis of an ARC reveals that a subset of the panel is altered, each point of that subset can be genetically screened by either compounds or additional genes that affect that specific point in the subset. The screening of additional genes against the original ARC phenotype, or any point in the PRIYSM panel subset, can establish genetic epistasis and identify novel members in the genetic pathways. In addition, compounds identified on the basis of ARC phenotype reversal can quickly be screened with the PRIYSM panel to determine if the compound directly counteracts the heterologous protein (such that all points in each ARC network are altered) or if the compound effects are indirect (affecting only a few points in the ARC network). Finally, since PRIYSM technology does not rely on a growth readout, heterologous genes that do not yield an altered growth phenotype can still be analyzed based on their effect with the PRIYSM panel.

What is claimed:

1. A method for determining genetic pathways in a host cell, comprising:
a) providing a plurality of first host cells;
b) transforming said first host cells with a plurality of different nucleic acid constructs, wherein said constructs comprise a plurality of different host cell genes, each operatively linked to a polynucleotide encoding an detectable label;
c) culturing said transformed cells under altered conditions sufficient to alter expression of a gene in said host cell; and
d) selecting cells which exhibit said label in response to said altered conditions.

2. The method of claim 1, wherein said host cells comprise eukaryotic cells.

3. The method of claim 2, wherein said eukaryotic host cells comprise yeast.

4. The method of claim 1, wherein said first host cells further comprise a heterologous gene.

5. The method of claim 4, wherein said heterologous gene is a human gene.

6. The method of claim 1, wherein said nucleic acid construct further comprises a selectable marker.

7. The method of claim 6, wherein said nucleic acid construct further comprises an affinity label.

8. The method of claim 7, wherein said nucleic acid construct encodes HA, GFP, and URA3.

9. The method of claim 1, wherein said plurality of host cell genes comprises at least 50% of the genes found in said host.

10. The method of claim 9, wherein plurality of host cell genes comprises at least 80% of the genes found in said host.

11. The method of claim 10, wherein said plurality of host cell genes comprises substantially all of the genes found in said host.

12. The method of claim 1, wherein said nucleic acid constructs are integrated into the host cell genome.

13. The method of claim 1, further comprising:
integrating said nucleic acid constructs into the genomes of said host cells.

14. The method of claim 1, further comprising:
providing a second host cell, comprising a heterologous gene; and
mating said first host cells and said second host cells.

15. The method of claim 14, wherein said heterologous gene comprises a human gene.

16. The method of claim 15, wherein said heterologous gene comprises a plurality of human genes.

17. The method of claim 1, wherein said altered conditions are selected from the group consisting of altered osmolarity, altered culture temperature, radiation, presence of virus, and presence of a chemical.

18. A nucleic acid construct for determining the effect of a heterologous gene on a selected host cell, comprising:
a) A host cell gene;
b) A detectable label operatively linked to said host cell gene; and
c) A selectable marker gene.

19. The nucleic acid construct of claim 18, further comprising an affinity label.

20. The nucleic acid construct of claim 18, further comprising a recombinase recognition site flanking each end of said construct.

21. The nucleic acid construct of claim 19, further comprising a recombinase recognition site flanking each end of said construct.

22. The nucleic acid construct of claim 21, wherein said recognition site is a cre-lox site.
